(19) United States
(12) Patent Application Publication
(10) Pub. No.: US 2008/0183124 A1
(43) Pub. Date: Jul. 31, 2008

(54) Method and device for treating breast implant encapsulation

(76) Inventor: Zachary E. Gerut, Hewlett, NY (US)

Correspondence Address: BERNARD MALINA, ESQ.;MALINA & ASSOCIATES, PLLC, Suite 501, 60 East 42nd Street, New York, NY, 10165, US

(21) Appl. No.: 12/012,629

(22) Filed: Feb. 4, 2008

Related U.S. Application Data
(63) Continuation-in-part of application No. 11/881,716, filed on Jul. 27, 2007.
(60) Provisional application No. 60/820,811, filed on Jul. 31, 2006.

(51) Int. Cl. A61B 18/14; A61M 37/00

(52) U.S. Cl. 604/21; 606/41

(57) ABSTRACT

A method and a surgical instrument are provided for treating breast implant encapsulation. The method involves creating an incision into the skin of a human chest, revealing a breast augmentation implant underneath the incision, placing a fibrin biomatrix in an area near the implant either concurrent with or subsequent to implantation, and dispensing concurrent with or subsequent to fibrin biomatrix placement a depository steroid or collagenase in the area near the implant to prevent or remove scar encapsulation.

### CROSS-REFERENCES

This application is a Continuation-in-Part of patent application Ser. No. 11/881,716, filed Jul. 27, 2007, entitled “Method and Device for Treating Breast Implant Encapsulation,” which claims priority from Provisional Patent Application Ser. No. 60/820,811, filed Jul. 31, 2006.

### BACKGROUND OF THE INVENTION

1. Field of the Invention

The invention concerns a method and device for treating breast implant encapsulation through minimal incision using local anesthesia.

2. Related Art

According to the statistics compiled by the American Society for Aesthetic Plastic Surgery, 383,866 breast augmentation procedures were performed during the year 2006 in the United States. This procedure is therefore the second most common operation performed by American plastic surgeons, only liposuction being more popular. The number of women undergoing this procedure has almost doubled since the year 2000 with an increase of approximately 30,000 patients per year since 2000.

Several millions of American women have undergone breast augmentation since the development of the procedure by Doctors Cronin and Gerow in the 1960's. Initially, only silicone filled implants were available for implantation. Saline filled implants have been used with more or less popularity over the decades. In 1991, when the Food and Drug Administration placed a moratorium on the use of silicone implants, the use of saline filled implants was of course instantly more popular. This popularity has remained steady. In 2006, more than 80% of implants used were saline filled. Very recently, the Food and Drug Administration released silicone implants for full and unrestricted use by plastic surgeons for their patients.

Any biocompatible implant placed within the human body, if not rejected or extruded, will be covered with a “shell” of scar tissue as part of the normal healing process. For solid implants such as pacemakers, chin implants or hip replacements, the scar shell is of no importance. For a malleable implant such as a breast implant, this scar shell can become a difficult problem if the scar tissue becomes thick and progressively tighter. This process is called capsular contraction. Capsular contracture results in dissatisfaction and deformity among the more than 2 million women who have had silicone implants. The literature describes contracture rates as high as 74% but most experts agree the incidence is roughly 35% for silicone implants and less for saline implants. It is also generally accepted that implants placed under the chest (pectoralis) muscle will have a lower incidence of capsular contraction than implants placed above the muscle. The etiology of capsular contraction is unknown. Various causal factors have been implicated but not proven including subclinical infection, postoperative bleeding resulting in a more than usual amount of blood surrounding the implant and the physical and/or chemical properties of the breast implant “bag” containing either silicone or saline filler.

Whatever the cause, capsular contracture can cause local discomfort and malposition and/or abnormal shape of the implant. The tightening scar tissue can eventually deform what should be a soft malleable breast implant into a hard round shape because the smallest surface area for any given volume is a sphere. It can also “pull” the implant into an abnormal position. Classification of the extent of contracture is highly subjective. Drs. Little and Baker (1980) developed a classification for the capsular contracture present following breast augmentation, and this is still the standard used for evaluation of this complication in patients. The grades of capsular contracture are divided into four types: (1) Grade I classification of capsular contracture of the augmented breast feels as soft as an unoperated breast; (2) Grade II capsular contracture is minimal contracture. The breast is less soft than an unoperated breast. The implant can be palpated but is not visible; (3) Grade III is moderate. The breast is firmer. The implant can be palpated easily. It may be distorted or visible; (4) Grade IV is severe. The breast is hard, tender and painful with significant distortion present.

At the present, the only nonsurgical treatment for capsular contraction is closed capsulotomy in which extreme manual pressure is applied to the implant to rupture the surrounding scar. Unfortunately, this can and sometimes does result in rupture of the implant. The only other treatment is surgical; either capsulotomy or capsulectomy. Capsulotomy is the smaller of the two procedures: a small (5 to 7 cm) skin incision is made, dissection is done to reach the implant and the scar tissue is cut, usually circumferentially to release the implant. Some surgeons perform a capsulectomy in which most of or the entire scar capsule is removed. Neither of these procedures have a high rate of success as the scar tissue will often reform.

In summary, capsular contraction represents a common problem amongst the millions of women with breast implants. The etiology is unknown and therefore is not reliably predictable. There is also no clear preventative treatment or curative treatment. For these reasons, it is evident that any improvement in prevention or treatment would be very welcome to plastic surgeons and to the population of patients who suffer from these very common issues.

### SUMMARY OF THE INVENTION

A method for treating breast implant encapsulation is provided which includes:
creating an incision into skin of a human chest;
approaching a breast augmentation implant underneath the incision;
placing a fibrin biomatrix in an area near the implant either concurrent with or subsequent to implantation; and
dispensing concurrent with or subsequent to fibrin biomatrix placement a depository steroid in the area near the implant to prevent or remove scar encapsulation or dispensing concurrent with or subsequent to fibrin biomatrix placement, the enzyme collagenase in the area near the implant to prevent or remove scar encapsulation.

Further, there is provided a surgical instrument to accomplish the method for treating breast implant encapsulation, the instrument including:
an elongate housing having distal and proximal ends;
an electrode within the housing and terminating at the distal end; and
a fluid transporting conduit within the housing delivering the fluid to the distal end through multiple discharge openings.

### BRIEF DESCRIPTION OF THE DRAWING

Further advantages and features of the present invention will become more evident through consideration of the drawing in which:

FIG. 1 is a cross-sectional view of a surgical instrument for use with the present method; and

FIG. 2 is a front view of the surgical instrument shown in FIG. 1 in a direction focusing on the distal end.

### DETAILED DESCRIPTION OF THE INVENTION

Now I have found an improved method for treating breast implant encapsulation known also as capsular contraction. The method requires placement of a fibrin biomatrix into an area near the implant. Further necessary is the dispensing concurrently or at a subsequent time, a depository steroid over the same area.

Fibrin biomatrix is essentially a fibrinogen aprotinin solution, which among other ingredients can contain Factor XIII, in highly concentrated form. Ordinarily a second component solution of thrombin and calcium chloride is applied with the fibrinogen aprotinin solution for purposes of cross-linking and coagulation. Commercially, the fibrin biomatrix is available from Baxter International, Inc. as a kit of the two solutions under the trademark TISSEEL VH.

The depository steroid for use in the present invention is a corticosteroid. Particularly preferred is Triamcinolone commercially available under such trade names as KENALOG, ARISTOCORT, NASACORT, TRI-NASAL, TRIDERM, and AZMACORT.

Collagenase is an enzyme that occurs naturally in the human body; its purpose is to break down collagen; collagen is the principal component of scar tissue and scar tissue causes breast implant encapsulation. Collagenase is commercially available from Biospecifics Technology Corporation.

It is believed that the advantage of the combination of materials is that the fibrin biomatrix helps distribute and avoids any pooling of the depository steroid or collagenase. High rates of complications are thought to result from pooling after placement of the steroid or collagenase. These are avoided through the scaffolding effect of the fibrin biomatrix.

For purposes of the present inventive method, it is advantageous to utilize a surgical instrument I have developed for treating the breast scar encapsulation problem.

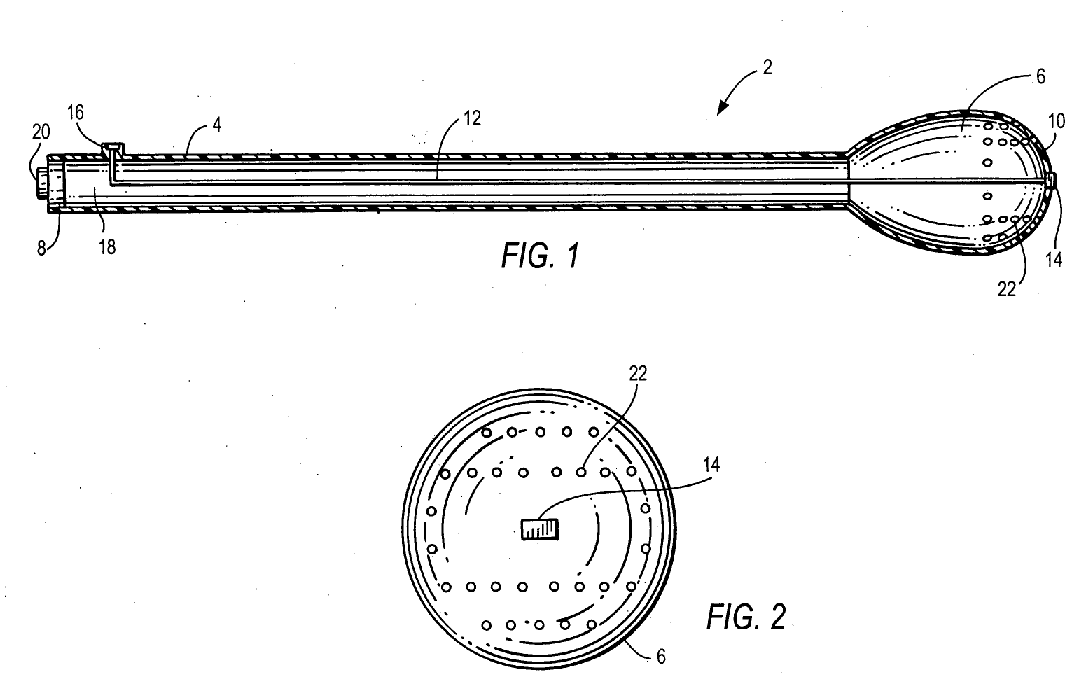

FIG. 1 illustrates a cross-sectional view of the instrument 2. This instrument includes an elongated housing 4 having a distal end 6 and a proximal end 8. The distal end terminates as a round blunt tip 10.

An electrode 12 traverses a length of the housing, is supported in plastic of the blunt tip, and protrudes slightly from the tip to expose an electrode end 14. Near the proximal end, the electrode 12 is connected at terminal 16 to an electrical source.

A fluid transporting conduit 18 traverses the housing. A fluid source is delivered into the conduit through a luer locking device 20 arranged on the proximal end of the instrument. Fluid transported down the conduit can exit the blunt tip 10 through a multiplicity of discharge openings 22. These openings or apertures may range in number from 2 to 100, preferably from 5 to 50. FIG. 2 illustrates one arrangement of the discharge openings.

Fluid to be transported through the conduit will be local anesthesia which includes the depository steroid or collagenase.

A typical medical procedure of the present invention is a follows. A small skin incision is made under local anesthesia. The surgical instrument is then inserted into subcutaneous tissue. As the instrument advances, the discharge openings at the blunt tip disperse the local anesthesia into the tissue and into the scar capsule. This avoids the need to bring a needle close to the implant thereby ensuring the implant does not become perforated. This also avoids the need for sedation or general anesthesia. The blunt protruding electrode end is energized to heatingly cauterize and dissect through the subcutaneous tissues and into the capsule thereby avoiding implant damage. Once the capsule is entered, the distal end of the instrument can be used to cut through the scar capsule thus releasing the implant and relieving the contracture and then it is moved around within the capsule to disperse the fibrin biomatrix laden with the depository steroid or collagenase. In a second embodiment of the invention, the fibrin biomatrix may in advance of the scar encapsulation treatment be initially introduced with the implant.

It will be understood that this specification is exemplary and the invention therein is to be liberally construed within the scope of the attached claims.

What is claimed is:

1. A method for treating breast implant encapsulation comprising:
creating an incision into skin of a human chest;
revealing a breast augmentation implant underneath the incision;
placing a fibrin biomatrix in an area near the implant either concurrent with or subsequent to implantation; and
dispensing concurrent with or subsequent to fibrin biomatrix placement a depository steroid or collagenase near the area to prevent or remove scar encapsulation.

2. The method according to claim 1 wherein the fibrin biomatrix is a fibrinogen aprotinin.

3. The method according to claim 2 wherein the fibrin biomatrix is delivered as a solution further comprising Factor XIII, thrombin and calcium chloride.

4. The method according to claim 1 wherein the depository steroid is triamcinolone.

5. The method according to claim 1 wherein the collagenase is a scar-treating enzyme.

6. The method according to claim 1 wherein the depository steroid is delivered as a fluid by a surgical instrument, the instrument comprising:
an elongate housing having distal and proximal ends;
an electrode within the housing terminating at the distal end; and
a fluid transporting conduit within the housing delivering the fluid to the distal end.

7. The method according to claim 5 wherein the distal end terminates as a round blunt tip from which protrudes an end of the electrode.

8. The method according to claim 6 wherein the fluid transporting conduit exits the tip through multiple discharge openings.

9. The method according to claim 5 further comprising dissecting a scar encapsulation by contact with the electrode concurrently with or prior to dispensing the depository steroid in the area near the implant.

10. A surgical instrument comprising:
an elongated housing having distal and proximal ends;
an electrode within the housing terminating at the distal end;
a fluid transporting conduit within the housing delivering the fluid to the distal end through multiple discharge openings.
